(19) United States
(12) Patent Application Publication
(10) Pub. No.: US 2002/0035739 A1
(43) Pub. Date: Mar. 21, 2002

(54) Evolution of plant disease response plant pathways to enable the development of based biological sensors and to develop novel disease resistance strategies

(76) Inventors: Michael Lassner, Foster City, CA (US); James English, Burlingame, CA (US); Gusui Wu, Davis, CA (US)

Correspondence Address: LAW OFFICES OF JONATHAN ALAN QUINE, P O BOX 458, ALAMEDA, CA, 94501

(21) Appl. No.: 09/849,452

(22) Filed: May 4, 2001

Related U.S. Application Data
(60) Provisional application No. 60/202,233, filed on May 5, 2000.

(51) Int. Cl. A01H 5/00; C12Q 1/68; G06F 19/00

(52) U.S. Cl. 800/279; 435/6; 702/20

(57) ABSTRACT

Methods for producing and identifying plant disease resistance (R) genes and elicitors with novel and desirable characteristics are provided. Methods for producing such R genes and elicitors using RNA recombination procedures are provided. Bio-detectors that are reporter genes responsive to induction by plant R genes, and the plant R genes that induce them are provided.

### CROSS REFERENCE TO RELATED APPLICATIONS

The present application claims priority to and benefit of United States Provisional Application 60/202,233, filed May 5, 2000, the disclosure of which is incorporated herein by reference in its entirety for all purposes.

### COPYRIGHT NOTIFICATION

Pursuant to 37 C.F.R. 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Disease resistance in plants is a trait with significant agronomic repercussions. Each year, millions of tons of food and other plant products are lost as a result of plant pathogens. Therefore, it is not surprising that a great deal of time and expense has been expended on efforts to engineer plants with improved disease resistance. Much of this effort has been focused on understanding and manipulating the innate defense mechanisms of important crop and experimental plant species.

Heritable genetic differences in susceptibility to pathogens are apparent in many plant populations, such that a given plant pathogen causes disease only in a sub-portion of naturally occuring populations. Resistant plants respond to pathogen infections with a variety of specific and non-specific defense mechanisms, including localized cell death, accumulation of antimicrobial compounds, and alterations in the cell wall. The molecular basis of these host defense mechanisms provides an attractive target for attempts to derive plants with improved resistance to pathogens, and other environmental stresses or stress inducing agents (stressors).

Detailed investigations into the interactions between plant pathogens and their hosts have revealed a gene-for-gene relationship between avirulence and resistance, in which single genes determining avirulence on the part of the pathogen, and resistance on the part of the plant host, interact in a pairwise manner. Upon invasion of a host plant, the pathogen secretes specialized molecules or elicitors that facilitate the infection process. Activation of host defense mechanisms is usually triggered by the recognition of an elicitor by the host plant. This initial recognition is the function of disease resistance genes or R genes.

In the presence of the appropriate R gene, the plant recognizes a specific elicitor, which in turn activates a number of signal transduction pathways that initiate an effective defense response. Cells at the invasion site exhibit changes in the phosphorylation state of molecular targets, (including second messengers such as kinases), ion fluxes and production of reactive oxygen species. These changes lead to a type of rapid and localized programmed cell death designated the hypersensitive response (HR). In addition, signals such as salicylic acid are induced which activate a systemic acquired resistance (SAR) response that confers broad resistance against subsequent infection by a wide variety of pathogens, in a nonspecific manner.

Typically, plant R genes act in a dominant fashion to confer effective and specific resistance to plant diseases caused by fungal, bacterial, viral and nematode pathogens. In the last several years, more than twenty R genes have been cloned from a variety of different plants. R genes usually recognize, and confer resistance, to a specific strain or race of a pathogen dependent on the presence of a specific avirulence gene (elicitor of resistance). Recent progress in understanding the structure of R gene products reveals remarkable structural similarities among them, although the pathogens to which they confer resistance (and the avirulence gene products they recognize) are very diverse.

Using R genes to generate novel and broad spectrum disease resistance has been a goal of plant biotechnology. However, because of the extreme specificity of these genes and the lengthy and costly process of making transgenic plants expressing R genes, it has been difficult to assess the functionality of individual R gene products, whether naturally occuring or mutant, or to evaluate the interactions of R and Avr proteins in planta. A simple and rapid method to produce and assess the function of R genes and to test the interactions between such R genes and elicitors is needed. The present invention addresses these and other needs, as will be apparent upon review of the following disclosure.

### SUMMARY OF THE INVENTION

The present invention provides methods for identifying and improving R genes and elicitors involved in plant defense responses. Plant defense responses include plant disease responses to pathogens, such as viral, bacterial, fungal, insect or nematode pathogens and pests, as well as responses to environmental stresses such as heat, drought, uv irradiation and wounding. One aspect of the present invention relates to methods for identifying plant disease resistance genes (R) with novel characteristics, e.g., novel elicitor interactions, kinase activation and downstream signalling. Embodiments of the invention provide methods of identifying such novel R genes by recombining R gene segments to produce a diversified library of R genes, and identifying among the library members R genes with the specified characteristic. Diversification is accomplished by a variety of nucleic acid recombination procedures, e.g., nucleic acid shuffling, in silico, in vitro, or in vivo, optionally in combination with one or more additional mutagenesis technique. In some embodiments, recombination, e.g., nucleic acid shuffling, is performed recursively, with or without, interspersed selection of desired products. In some embodiments, RNA “shuffling” is performed in vitro in plant cells.

Identification of R genes with characteristics of interest is performed by expressing the R gene product in a plant cell, and screening for improved traits, or other desirable outcomes. Expression occurs following stable integration of the recombinant R gene operably linked to a functional promoter, or via cytoplasmic expression after introduction of the recombinant R gene via a non-integrating viral vector. Such vectors include both RNA and DNA viruses, e.g., tobamoviruses, potexviruses, potyviruses, tobraviruses, and geminiviruses. In some embodiments expression is regulated by a viral subgenomic promoter. In other embodiments, the recombinant R gene is introduced to the plant via infection with a plant pathogen, such as a bacterial pathogen, that transfers the recombinant R gene, optionally including a target signal, according to pathogen infection mechanisms into the plant cell.

In some embodiments, a the plant cell expressing the R gene is exposed to an elicitor of a plant defense response, such as the product of a Avr gene or gene homolog. In alternative embodiments, the elicitor is provided by a plant pathogen, or by a non-pathogenic microorganism or virus. In some embodiments, the non-pathogenic microorganism is a species of Pseudomonas. Alternatively, the plant cell expressing the R gene is a transgenic plant cell that expresses an Avr gene.

Interactions between the R gene and the elicitor are detected by a variety of screening protocols useful for detecting a disease response or molecular or biochemical event associated with a disease response. In some embodiments, disease resistance is evaluated based on observations of a decrease in symptoms or pathogen growth. In other embodiments, hypersensitive responses (HR), systemic acquired resistance (SAR) responses, induction of genes associated with the HR or SAR, or an accumulation of gene products or other compounds associated with the HR or SAR.

In some embodiments, the novel R genes identified according to the methods of the invention are recovered, e.g., by PCR, LCR, Qβ-amplification, cloning, isolation of RNA transcripts and/or reverse transcription. In some embodiments, the recovered R genes are stably integrated into plant cells, and the plant cell optionally regenerated to produce transgenic plants. Transgenic plants so-produced are a feature of the invention.

The invention further provides methods for identifying elicitors of plant defense responses with desired properties. Such methods involve recombining nucleic acids encoding peptide or protein elicitors or encoding enzymes catalyzing the production of elicitors, including complex biological molecules, e.g., cell wall components, carbohydrates, etc., as well as small molecule elicitors, and their gene homologs and exposing plant cells to the elicitor expressed by the recombined gene or synthesized by the recombined enzyme. Following exposure, a plant disease response is detected, facilitating identification of elicitors with desired properties.

In some embodiments, recombination of Avr genes and Avr gene homologs is performed by nucleic acid shuffling, in silico, in vitro, or in vivo. Optionally, shuffling is performed recursively. In some embodiments, the shuffling is performed in plants using RNA viral vectors comprising the Avr or other genes of interest.

The plant cell is exposed to an elicitor either by external application, or by expression by a viral vector or pathogen to which the plant is exposed. In preferred embodiments, the viral vectors are non-integrating viral vectors, including RNA and DNA plant viruses. In some cases the plant cell is a transgenic plant cell that expresses an R gene of the invention.

In some embodiments, the nucleic acid encoding the elicitor or enzyme catalyzing production of an elicitor with a desired property is recovered, and optionally introduced and stably integrated into a plant cell. Optionally, the transgenic plant cell is regenerated to produce a transgenic plant. Such transgenic plants are also a feature of the invention.

The invention also provides methods for identifying functional interactions between plant disease resistance genes and elicitors involving introducing a plant disease resistance (R) gene and an elicitor, or enzyme catalyzing production of an elicitor, into a plant cell and detecting a plant defense response induced by their interaction. In an embodiment, the R gene and the elicitor are introduced by viral vectors that express the R gene and the elicitor cytoplasmically in the host plant cell. In another embodiment, the R gene is introduced into the cell by a plant pathogen expressing the elicitor. Optionally, the R gene includes a targeting signal, and/or is translocated from the pathogen to the plant cell by a secretory system, such as a Type III secretory system of the plant pathogen. In some embodiments, the R gene and/or the gene encoding an elicitor or enzyme catalyzing production of an elicitor are recombinant, e.g., shuffled, genes.

Another aspect of the invention relates to methods of producing genes, including R genes and Avr genes with desired properties. A plurality of RNA viral vectors containing genes of interest are introduced into a cell, and the cells are grown under conditions permitting replication and recombination of the viral sequences. Optionally, the viral vectors are recovered, and the recombination is performed recursively. After recombination of the viral RNA molecules, a viral vector comprising a gene with a desired property is identified. The viral vectors are introduced into cells by inoculating the cell with infectious RNA transcripts. Alternatively, a plurality of cDNA molecules corresponding to viral transcripts are used to introduce the genes of interest into the cell. In the latter case, the plurality of cDNA molecules can be introduced by a variety of techniques including, electroporation, microinjection, biolistics, agrobacterium mediated transformation or agroinfection. In preferred embodiments, the RNA viral vectors are plant virus vectors, and the cells are plant cells. Such vectors include, tabamoviruses, potyviruses, tobraviruses, and potexviruses. In some embodiments, the plant cells are isolated cells grown in culture. In other embodiments, the plant cells are plant protoplasts, plant tissues, plant organs or intact plants.

In an embodiment, two viral vectors having complementary mutations in proteins involved in systemic infection are used to introduce nucleic acids comprising genes of interest. Upon recombination, infectivity is restored, thereby facilitating selection of recombinant genes of interest. Exemplary proteins involved in systemic infection include viral coat proteins and viral movement proteins.

The invention further provides for bio-detectors for sensing environmental stresses, including invasion by pathogens. The bio-detectors of the invention comprise an R gene encoding a product capable of activation by an elicitor, and a reporter, such as a visual reporter, regulated by a promoter, such as the promoter of a pathogenesis related (PR) gene, that is responsive to activation by the product of the R gene. In an embodiment, the R gene is a recombinant, e.g., shuffled, R gene with a specified characteristic of the invention. In another embodiment, the elicitor is the product of a recombinant, e.g., shuffled, Avr gene.

Transgenic plant cells and transgenic plants comprising the nucleic acids of the invention are also a feature of the invention. Similarly, the use of the nucleic acids of the invention as bio-detectors, or to confer resistance in stably or transiently transfected plants is a feature of the invention.

### BRIEF DESCRIPTION OF THE FIGURES

FIG. 1 is a schematic illustration of a viral vector of the invention.

### DETAILED DISCUSSION OF THE INVENTION

The present invention relates to the elucidation and manipulation of components of plant disease responses. Adaptive plant disease responses which serve to protect a plant from pathogenic or environmental insults are initiated through the interaction between plant disease resistance (R) genes and environmental or pathogen-derived elicitors. Such interactions are typically highly specific based on a ligand/receptor like interaction between an elicitor and the product of an individual R gene. The ability to manipulate or engineer the interactions between elicitors and the products of plant disease resistance genes would be of significant value. For example, methods for improving or altering the specificity of R genes, e.g., to increase the number and/or type of elicitors recognized, or to provide novel elicitor specificities are highly sought after (see, e.g., Brande et al. (2001) Plant Cell 13:255-272; Renier et al. (2001) Plant Cell 13:273-285; and WO 00/078944 “Methods to design and identify new plant resistance genes” by Scofield, published Dec. 28, 2000), and offer such varied benefits as increased crop yield, improved environmental range, resistance to heat or draught, reduction in pesticide use, among many others.

The methods described herein provide various means for generating and identifying plant disease resistance (R) genes and elicitors with novel and desirable properties, as well as functional interactions between resistance genes and elicitors. The methods described herein offer the means to identify and manipulate the components of plant disease response pathways to produce plants with enhanced disease resistance traits. Directed evolution processes are used to develop plant disease resistance, “R” genes and elicitors with a variety of novel and desirable characteristics. In particular, methods of diversifying DNA and RNA, e.g,. by shuffling, are described that enable the production and selection of R genes with novel elicitor specificities, multi-elicitor specificities, improved signalling capabilities, and the like, as well as the production of novel elicitors with desired properties. In particular, novel methods for recombining substrate nucleic acids using RNA viral vectors in planta are described. Such methods offer a rapid and convenient means of diversifying and screening R genes, and/or elicitors in vivo, e.g., in a target plant of interest.

Methods for conferring resistance to plant pathogens by stably or transiently expressing such artificially evolved R genes and elicitors are also described. In addition, the present invention also relates to the use of novel R genes and associated signalling pathways as bio-detectors of plant pathogens and other environmental stressors.

Definitions

Unless defined otherwise, all scientific and technical terms are understood to have the same meaning as commonly used in the art to which they pertain. For the purpose of the present invention, the following terms are defined below.

“Nucleic acid shuffling” refers to an artificial process of recombination between nucleic acid molecules, in vitro, in vivo, or in silico, for the purpose of generating diversity in a nucleic acid population. “DNA shuffling” and “RNA shuffling” refer to such recombination in populations of DNA and RNA molecules, respectively. According to some formats, recombination is homology based, e.g., certain in vitro and in vivo shuffling methods, while alternative formats, e.g., in silico shuffling, do not require sequence similarity to generate recombinant, i.e., “shuffled” sequences. In many instances, nucleic acid shuffling is performed recursively by repeating the recombination process one or more times.

Nucleic acid shuffling is typically employed in conjunction with one or more screening or selection procedures in a process of “directed evolution,” that is, evolution of nucleic acid sequences or phenotypes to acheive a predetermined outcome, such as a specified characteristic or other desired property.

In the context of the present invention, a “gene of interest” can be essentially any nucleic acid sequence, e.g., DNA or RNA, or representation thereof, e.g., character strings in a computer readable medium. Genes of interest include, for example, sequences encoding proteins of interest, e.g., R gene products, enzyme, elicitors, regulatory sequences such as promoters and enhancers, gene homologs and pseudogenes.

A gene “fragment” or gene “segment” is any subportion of, up to and including, an entire gene, or nucleic acid incorporating the gene, e.g., vector, virus, episome, chromosome, etc. A gene fragment or segment can also be a synthesized nucleic acid such as an oligonucleotide corresponding to a gene or gene homolog, or a character string representing a gene or gene homolog in silico. As a result, e.g., of nucleic acid shuffling, gene fragments or segments are recombined to form “recombinant gene fragments” or “recombinant gene segments.” According to the format selected, the recombinant gene segments are DNA, RNA or, e.g., an in silico representation thereof.

“Screening” is, in general, a two-step process in which one first determines which cells, organisms or molecules (e.g., nucleic acids, proteins, etc.), do and do not express a detectable marker, or phenotype (or a selected level of marker or phenotype), and then physically separates the cells, organisms or molecules, having the desired property or characteristic. “Selection” is a form of screening in which identification and physical separation are achieved simultaneously by expression of a selectable marker, which under some circumstances, allows cells expressing the marker to survive while other cells die (or vice versa). Screening reporters include luciferase, β-glucuronidase, green fluorescent protein (GFP), carotenoid biosynthetic enzymes, and anthocyanin regulatory genes (e.g., the maize Lc gene). Selectable markers include antibiotic and herbicide resistance genes. A special class of selectable markers are negatively selectable markers. Cells or organisms expressing a negatively selectable marker die under appropriate selection conditions while organisms lacking or having a non-functional form of the marker survive. Examples of negatively selectable markers useful in the context of plant genetic engineering include a number of genes involved in herbicide metabolism, including: dlh1, codA, tms2 and NIA2

As used herein, a “plant pathogen” is any organism or agent resulting in the infection of a plant or plant tissue. Common pathogens include viruses, bacteria, fungi, insects and nematodes.

A “plant defense response” refers to a response by a plant to an environmental stress. Such a response can be a “plant disease response” to an infectious agent or plant pathogen, but can also include plant responses to environmental stresses caused by ultraviolet irradiation, heat, drought, wounding, and the like.

A “plant disease resistance gene” or “R” gene is a genetic determinant of specific pathogen resistance. For purposes of the present disclosure, the product encoded by an R gene is referred to alternatively as a “product of an R gene,” or an “R protein.”

An “elicitor” of resistance is a composition that interacts with a product of an R gene. An elicitor can be a protein or peptide gene product, e.g., a product of an Avr (avirulence) gene or Avr gene homolog, or a small molecule, or compound produced by an enzyme product of an Avr gene or a biochemical pathway comprising such an enzyme.

A “hypersensitive response” (HR) involves the rapid, localized death of host cells in response to pathogen challenge. A “systemic aquired resistance” (SAR) response, is the result of systemic signals, e.g., salicylic acid, activated by pathogenic challenge, that confer nonspecific resistance against subsequent infection. Both responses generally involve the activation of signalling pathways, resulting in the induction of genes, and the accumulation of gene products associated with the HR or SAR, respectively.

“Resistance to infection” refers to a decreased susceptibility to a pathogenic challenge. Such resistance can be measured as a decrease in symptoms or as a decrease in pathogen growth following exposure.

As used herein, a “plant cell” refers to an isolated plant cell, e.g., a plant cell maintained in suspension culture, as well as a plant protoplast, plant tissue, plant organ, whether isolated or intact, or an intact plant.

A “bio-detector” refers to a molecular system, typically a receptor/activator capable of interacting with an environmental cue, e.g., an endogenous or exogenous ligand representative of an environmental or physiological state, and a responder/reporter giving rise to a detectable alteration in state, e.g., induction of gene expression.

Introduction

In response to numerous biotic and abiotic stresses, plants mount a variety of defense responses that are both localized and systemic in their action. In general, these fall into two broad categories: non-specific responses that serve to protect the plant against a variety of agents and insults, and specific host responses that involve the interaction a particular pathogen and its host.

Non-specific responses include alterations in protein composition due to both gene induction and modifications in existing proteins, as well as structural changes in the organization of the cell wall. For example, physical injury, whether accompanied by invasion by a pest or pathogen, or due simply to mechanical trauma, results in rapid changes in the architecture of the cell wall. Loss of cellular integrity induces callose synthase activity, increasing synthesis of the β1,3 glucan polysaccharide, callose. In addition to changes in cell wall constituents, changes in organization are also observed, including hydrogen peroxide mediated cross-linking of cell wall proteins.

Cellular damage also results in membrane depolarization in surviving cells, accompanied by alterations in ion fluxes and second messenger signalling pathways resulting in adaptive metabolic changes. For example, mitochondrial electron transport is inhibited, and free fatty acids become substrates for α-oxidation, providing an alternative to carbohydrates as a short term energy source.

Localized increases in plant growth regulators, such as ethylene, and increases in cell division are also observed in some instances. In other cases, induction of enzymes such as phenylalanine amonia-lyase (PAL) involved in the lignification of plant cell1 walls, play a significant role at a local level, to prevent further damage to the plant.

Multiple changes are also seen at a systemic level. For example, expression of certain genes is induced (e.g., serine proteinase inhibitors, pin1 and pin2), not only locally, but at sites distant from the site of wounding. Induction of gene activity at sites distant from the wound involves long-range signalling events, that depending on the circumstance involve chemical, ionic, and hydrostatic mechanisms. These non-specific defense responses are activated by a wide range of environmental stimuli, including heat, ultraviolet irradiation, drought, and exposure to ozone.

In addition to such non-specific plant defense responses, specific responses, e.g., to individual pathogens, also play a role in protecting the plant from attack, particularly by pathogens. Such specific host resistance responses are governed by one, or a few genes, activated in response to an attempted infection. First proposed in the 1950's, the gene-for-gene hypothesis of host specificity, (Flor (1955) Phytopathology 45:680), proposes that corresponding genes for resistance, “R” genes, and avirulence, “A” genes, exist in the plant and pathogen, respectively. According to the traditional view, an “incompatible” interaction between R and A genes resulted in resistance through induction of the plant hypersensitive response (HR). Conversely, a susceptibility resulted from a “compatible” interaction between R and A genes.

The plant hypersensitive response (HR) is initiated upon attempted infection by an avirulent pathogen strain by a rapid oxidative burst resulting in the accumulation of hydrogen peroxide (H₂O₂) and active oxygen radicals, (e.g., O₂⁻). In addition to driving cross-linking of cell wall structural proteins, H₂O₂ stimulates a rapid influx of Ca²⁺ ions. Membrane depolarization, associated with Calcium ion flux triggers a protein kinase cascade activating a physiological cell death program. Similar to apoptosis in animal cells, programmed cell death of the plant HR results in fragmentation of DNA, and characteristic alterations in cell-morphology, e.g., plasma membrane blebbing, cell shrinkage, and nuclear condensation.

Concommitant, with Ca²⁺ dependent signalling, H₂O₂ accumulation also results in the induction of genes, e.g., glutathione-S-transferase, glutathione peroxidase, having cellular protectant function in adjacent cells.

In a separate signalling pathway, induction of salicylic acid, e.g., by H₂O₂, leads to systemic acquired resistance (SAR), which provides broad-spectrum protection against a wide range of pathogens. The SAR response is characterized by the induction of specific gene products, including proteins with antimicrobial activity. For example, induction of β1,3 glucanase, the product of the Pathogenesis related (PR) gene BGL2 is characteristic of the SAR in a number of plants, including, e.g., tobacco and tomato. While a number of PR genes, characterized as acidic PR proteins, are effectively induced by salicylic acid, a second category of basic PR gene products is inducible by pathogens but not by salicylic acid alone, suggesting that additional pathways regulated by pathogen invasion contribute to resistance.

Plant Disease Resistance (R) Genes

Specific interactions between plant pathogens and their hosts are initiated via, typically, a gene-for-gene interaction between the product of a plant R gene and a pathogen-derived elicitor. Molecular characterization of a number of R genes has revealed that nearly all R gene products share a common structural motif, designated “leucine-rich repeats” (LRRs). Structure/function analyses of several R gene products have shown that the LRRs of R proteins function as receptors for interacting with pathogen-derived elicitor, and act as the specifictiy determinants of host-pathogen recognition. It is postulated that specific binding between the LRRs of an R protein and an elicitor triggers signal transduction via a kinase cascade, leading to eventual expression of defense genes such as PR genes. For example, in the case of the Xa21 gene product of rice, the LRR domain and a serine-threonine kinase domain are present within a single polypeptide of an R protein. The present invention describes methods for producing R genes and their products with novel specificities and signalling properties as well as methods of utilizing these “recognition-to-activation” systems in plants to evolve biogical detectors for both novel disease resistance and other applications.

An important feature of LRRs is their structural plasticity that enables them to interact with ligands of various origins and structural characteristics. By using nucleic acid diversification and screening or selection procedures, e.g., nucleic acid shuffling, to evolve the LRR domains of disease resistance genes, novel recognition specificities not found in nature, or not found in a given host in nature, are engineered into receptors capable of interacting with a wide variety of ligands.

One class of ligands particularly suited to the methods of the present invention, are components of crop pathogens of interest. Diversification of R genes, e.g., by nucleic acid shuffling of R genes and gene homologs, in combination with in vitro, and in vivo selection methods for detecting favorable interactions between diversified R genes and elicitors of interest, provides the means for developing robust resistance to pathogens for which innate specific resistance is absent or weak in a natural population. Evolved R genes, produced by these methods provide a means of conferring resistance to pathogens and, as described below, for detecting infection in natural and cultivated populations.

The evolved R genes with novel disease recognition properties are stably integrated into plant genomes as a transgene to produce disease resistant plants capable of vertical transmission of the disease resistant trait. Alternatively, evolved R genes can be delivered by viral vectors to trigger plant disease response pathways in a transient manner. The latter approach provides the benefit of facilitating treatment of plants growing in the field for pathogen infestations or infections that are discovered after planting, and for which the plants do not have endogenous disease recognition abilities. Other ligands include, but are not limited to, human and animal pathogens as well as other chemical ligands.

As well as producing R gene encoding products with novel recognition specificities, the methods of the invention provide ways to functionally modify kinase or other functional domains, altering signalling pathways that are triggered in response to the ligands. This provides an approach to enhancing or potentiating alternatively regulatable aspects of the HR and SAR responses, e.g., acidic and basic PR proteins.

Currently, there are more than 20 R genes cloned from different plant species (e.g., tomato, rice, barley, corn, soybean, flax, sugar beet, wheat and Arabidopsis). Many of the identified R genes are members of large gene families, which provide excellent pools of candidate genes for artificial evolution, e.g., by nucleic acid shuffling, because members of each gene family usually have relatively high sequence homology as well as ample diversity. Examples of suitable starting materials are provided in Table 1. However, it will be readily understood that the invention is equally applicable to any other cloned R genes and even uncloned and/or uncharacterized R genes, through modifications that will be readily apparent to those of skill in the art, and which are described herein and in the cited references.

For example, members of libraries, such as genomic libraries, various cDNA libraries, including expression libraries, and EST libraries comprising R genes, gene homologs and gene fragments are all suitable substrates for the methods of the invention. If desired, one or more enrichment step can be performed to increase the frequency of sequences with desired characteristics based on structural or sequence similarity with selected R genes, e.g., hybridization, PCR, etc.

TABLE 1
Exemplary cloned R genes.

| Gene Name | Genbank Accession Number |
|---|---|
| Bs2 | AF202179 |
| Cf2 | U42444 |
| Cf4 | AW035254 |
| Cf9 | AF053993 |
| Hcr2 | AF053994 |
| Hcr9 | AF119040 |
| Xa21 | E17291 |
| Rp1-D | AF107293 |
| Rpp5 | AC180943 |
| Rpp8 | AF089710 |
| RPM1 | AF122982 |
| RPS2 | U12860 |
| RPS4 | AJ243468 |
| PRF | U65391 |
| L6 | U27081 |
| M | U73916 |
| I2 | AF118127 |
| N | U15605 |
| Rx | AJ011801 |
| Mi | AF091048 |
| Dm3 | AH007213 |
| Xa1 | E17291 |
| Pib | AB013448 |
| Pto | U59315 |
| Pti1 | U28007 |
| Mlo | Z83834 |
| Hs1^{pro-1} | U79733 |
| LRK10 | U51330 |
| Fen | U59318 |

Elicitors of Resistance

The present invention provides methods for producing elicitors, whether protein or peptide products of Avr genes, or the products of enzymatic activities encoded by Avr products, with desired properties. Artificial evolution, e.g., nucleic acid shuffling, techniques are used to produce Avr genes that encode proteins with desirable properties. Such properties include but are not limited to, e.g., increased affinity of interaction with a specified R gene product, improved stability properties, improved transmission properties, and the like. Selection of Avr gene with desired properties is, as described, supra, by any of a variety of procedures for detecting functional interactions between elicitors and R gene products.

Elicitors of resistance, or “elicitors” are, broadly speaking, the primary or secondary products of pathogen avirulence (Avr) genes. An Avr gene is described as a genetic locus in a plant pathogen that determines race/cultivar-specific expression of disease resistance in conjunction with the functionally complementary R gene in a host. Such elicitors fall into two broad categories, both of which are targets for the methods of the present invention.

The first of these categories includes protein or peptide elicitors encoded, e.g., by pathogen Avr genes or Avr gene homologs. For example, “elicitins” are highly conserved protein elicitors produced by phytophthora and related fungal species, and can be further sub-divided into acidic (e.g., cinnamomin) and basic (e.g., cryptogein) elicitin groups. Another major group of protein elicitors are the “harpins” encoded by a subset of ORFs of the hrp operons (e.g., in Pseudomonas sp). Other ORFs of hrp operons encode components of a specialized secretory system required for transmission of harpins and other cellular components required for infection, and/or resistance, i.e., the so-called Type III secretory system. For example, harpinPss, the product of the P. syringae hrpZ gene, is a 34.7 kd extracellular protein containing two directly repeated sequences of GGGLGTP and QTGT that are necessary and sufficient for elicitor activity (He et al. (1993) Cell 73:1255). The AVR4 and AVR9 elicitors of the tomato pathogen Cladosporium fulvum are peptide elicitors or 28 and 106 amino acids that induce HR in tomato plants carrying the complementary Cf4 and Cf9 resistance genes, respectively.

The second category of elicitors includes small molecules synthesized as products of an enzymatic activity or biochemical pathway encoded by Avr genes. For example, cell wall breakdown products such as oligogalactonaturides and syringolide are produced upon initiation of infection, e.g., by activity of pathogen encoded endopolygalacturonase.

Directed Evolution of R/Avr Genes

A variety of diversity generating procedures, including nucleic acid shuffling protocols, e.g., multi gene shuffling protocols, are available and fully described in the art. The following documents describe a variety of recursive recombination and other procedures which can be used to diversify plant R genes and various genes related to the production, either directly or indirectly, of elicitors by the methods of the invention. The procedures can be used separately, and/or in combination to produce one or more variants of a nucleic acid or set of nucleic acids, as well variants of encoded proteins, e.g., R genes and proteins, Avr genes and proteins, etc. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the diversity generating procedures described herein can be the generation of one or more nucleic acids, which can be selected or screened for nucleic acids with or which confer desirable properties, or that encode proteins with or which confer desirable properties. Following diversification by one or more of the methods herein, or otherwise available to one of skill, any nucleic acids that are produced can be selected for a desired activity or property, e.g., the ability to induce one or more localized or systemic responses which confer pathogen resistance. This can include identifying any activity that can be detected, for example, in an automated or automatable format, by any of the assays in the art, e.g., visual or molecular assessment of pathogen resistance, measure of pathogenesis related gene induction, reporter induction, etc., (see below, “POST-RECOMBINATION SCREENING TECHNIQUES”). A variety of related (or even unrelated) properties can be evaluated, in serial or in parallel, at the discretion of the practitioner.

Descriptions of a variety of diversity generating procedures for generating modified R and/or Avr nucleic acids are found in the following publications and the references cited therein: Soong, N. et al. (2000) “Molecular breeding of viruses” Nat Genet 25(4):436-439; Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors: an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13: 3305-3316), double-strand break repair (Mandecki (1986) “Oligonucleotide-directed double-strand break repair in plasmids of Escherichia coli: a method for site-specific mutagenesis” Proc. Natl. Acad. Sci. USA, 83:7177-7181; and Arnold (1993) “Protein engineering for unusual environments” Current Opinion in Biotechnology 4:450-455). Additional details on many of the above methods can be found in Methods in Enzymology Volume 154, which also describes useful controls for trouble-shooting problems with various mutagenesis methods.

Additional details regarding various diversity generating methods can be found in the following U.S. patents, PCT publications and applications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination;” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” PCT/US00/26708 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Affholter.

In brief, several different general classes of sequence modification methods, such as mutation, recombination, etc. are applicable to the present invention and set forth, e.g., in the references above. That is, sequences corresponding to R and/or Avr genes can be diversified, by any of the methods described herein, e.g., including variuos mutation and recomibination methods, individually or in combination, to generate nucleic acids with specified characteristics relating pathogen resistance.

The following exemplify some of the different types of preferred formats for diversity generation in the context of the present invention, including, e.g., certain recombination based diversity generation formats.

Nucleic acids can be recombined in vitro by any of a variety of techniques discussed in the references above, including e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants is described in several of the references above, e.g., in Stemmer (1994) Proc. Natl. Acad. Sci. USA 91:10747-10751. Thus, R genes, Avr genes, domains (e.g., LRR domains) or other subsequences thereof, can be recombined in vitro to generate R genes and Avr genes with desirable (e.g., specified) characteristics.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above. Thus, nucleic acids corresponding to R genes, and Avr genes can be recombined within cells to provide a more diverse population of nucleic acids encoding R proteins, and Avr encoded elicitors (or biosynthetic enzymes involved in elicitor production) from which R proteins and elicitors with desirable properties can be isolated.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details on such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used, in which oligonucleotides corresponding to targets of interest (e.g., including one or more R gene, LRR domain, or subsequence therof) are synthesized and reassembled in PCR or ligation reactions which include oligonucleotides which correspond to more than one parental nucleic acid, thereby generating new recombinant R genes or Avr genes. Oligonucleotides can be made by standard nucleotide addition methods, or can be made, e.g., by tri-nucleotide synthetic approaches. Details regarding such approaches are found in the references noted above, including, e.g., WO 00/42561 by Crameri et al., “Olgonucleotide Mediated Nucleic Acid Recombination;” PCT/US00/26708 by Welch et al., “Use of Codon-Varied Oligonucleotide Synthesis for Synthetic Shuffling;” WO 00/42560 by Selifonov et al., “Methods for Making Character Strings, Polynucleotides and Polypeptides Having Desired Characteristics;” and WO 00/42559 by Selifonov and Stemmer “Methods of Populating Data Structures for Use in Evolutionary Simulations.”

In silico methods of recombination can be effected in which genetic algorithms are used in a computer to recombine sequence strings which correspond to homologous (or even non-homologous) seuqences corresonding to R and/or Avr genes. The resulting recombined sequence strings are optionally converted into nucleic acids by synthesis of nucleic acids which correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in WO 00/42560 by Selifonov et al., “Methods for Making Character Strings, Polynucleotides and Polypeptides Having Desired Characteristics” and WO 00/42559 by Selifonov and Stemmer “Methods of Populating Data Structures for Use in Evolutionary Simulations.” Extensive details regarding in silico recombination methods are found in these applications. This methodology is generally applicable to the present invention in providing for recombination of plant defense related nucleic acids in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library(ies) is annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “Single-Stranded Nucleic Acid Template-Mediated Recombination and Nucleic Acid Fragment Isolation” by Affholter, PCT/US01/06775.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences which hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods) to generate a more diverse set of recombinant nucleic acids.

Mutagenesis employing polynucleotide chain termination methods have also been proposed (see e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See, also, Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44.

Mutational methods which result in the alteration of individual nucleotides or groups of contiguous or non-contiguous nucleotides can be favorably employed to introduce nucleotide diversity into R genes and/or Avr genes, or subsequences thereof. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin & Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave & Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g. a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “METHODS FOR GENERATING AND SCREENING NOVEL METABOLIC PATHWAYS,” and Thompson, et al. (1998) U.S. Pat. No. 5,824,485 METHODS FOR GENERATING AND SCREENING NOVEL METABOLIC PATHWAYS) and their use to identify protein activities of interest has been proposed (In addition to the references noted above, see, Short (1999) U.S. Pat. No. 5,958,672 “PROTEIN ACTIVITY SCREENING OF CLONES HAVING DNA FROM UNCULTIVATED MICROORGANISMS”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods herein described.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215: 471) prior to diversifying according to any of the methods described herein.

Libraries can be biased towards nucleic acids which encode proteins with desirable enzyme activities. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “PRODUCTION OF ENZYMES HAVING DESIRED ACTIVITIES BY MUTAGENESIS.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

It will readily be appreciated that any of the above described techniques suitable for enriching a library prior to diversification can also be used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the format which is used, the nucleic acids of the invention can be recombined (with each other or with related (or even unrelated) nucleic acids to produce a diverse set of recombinant nucleic acids, including, e.g., R genes encoding proteins with novel and desirable functions, Avr genes encoding or involved in synthesizing elicitors with desired properties.

Following diversification, any nucleic acids which are produced can be selected for a desired activity. In the context of the present invention, this can include testing for and identifying any activity that can be detected, including in an automatable format, by any of the assays in the art. A variety of related (or even unrelated) properties can be assayed for, using any available assay. Exemplary screening methods are described below.

In one aspect, the present invention provides for the recursive use of any of the diversity generation methods noted above, in any combination, to evolve nucleic acids or libraries of recombinant nucleic acids that are involved in plant pathogen defense responses, e.g., R and Avr genes, genes encoding components of downstream signalling pathways, PR genes, genes inducible by an interaction between an R protein and an elicitor, and the like. In particular, as noted, the relevant nucleic acids which participate, or which putatively participate, in one or more defense response can be modified before selection, or can be selected and then recombined, or both. This process can be reiteratively repeated until a new or improved nucleic acid having (or conferring) a desired property or trait is obtained.

RNA Shuffling

In addition to the diversity generating, e.g., nucleic acid shuffling, methods described above, the present invention specifically provides a format for in vivo RNA reombination, e.g., “shuffling,” that is favorably employed in the generation of, e.g., novel R and/or Avr genes. Nucleic acids encoding, e.g., R genes, R gene homologs, LRR domains, or subsequences thereof are inserted into RNA viral vectors. In the context of diversifying plant related sequences, or sequences such as Avr genes that have a site of action in plants, plant viruses are the vector of choice. However, it will be understood that any type of RNA virus can be employed depending on the application. Selection of an appropriate viral vector is within the discretion of the practitioner and can largely be determined by the cell type wherein expression is desired and/or by the mode of action or site of action of the gene of interest.

In some instances it will be desirable to insert cDNA or other DNA sequences of interest into a DNA transcription vector capable of giving rise to infectious viral RNA transcripts. The methods for so doing are well established in the art, and referenced below. For example, cDNAs, oligonucleotides, genomic fragments, or other sequences encoding R proteins, or subportions of R proteins, or inactive or active gene homologs that are R gene related, can be cloned into reverse transcribed, double stranded viral cDNA molecules, which are optionally components of autonomously replicating vectors such as plasmids, episomes, T-DNAs, transposons, and the like.

In either case, a population of viral vectors, each comprising a variant of the gene of interest, is introduced into plant cells or tissues such that a single plant cell or tissue receives multiple different variants of the gene of interest. If infectious transcripts are used, following inoculation, RNA transcripts are cytoplasmically replicated under the control of viral replication sequences located, typically, within the 5′ terminal region of the transcript. Alternatively, after introduction, e.g., by electroporation, microinjection, or agrobacterium mediated transformation, the cDNA vector gives rise to RNA transcripts, which are then replicated in the cytoplasm of the cell by the viral RNA polymerase.

Both homologous and non-homologous recombination occur in RNA viruses, and both processes are believed to be mediated by template switching of the viral RNA-dependent RNA polymerase during replication. Specific mutations have been identified within viral RNA polymerases that affect the frequency of homologous or non-homologous RNA recombination. Accordingly, the RNA polymerase can be selected to bias the recombination process to acheive the desired outcome with respect to diversity generation. Alternatively, RNA shuffling as described herein, or other nucleic acid diversification, e.g., shuffling, methods can be used to derive viral RNA polymerases with enhanced homologous and/or non-homologous RNA recombination activity.

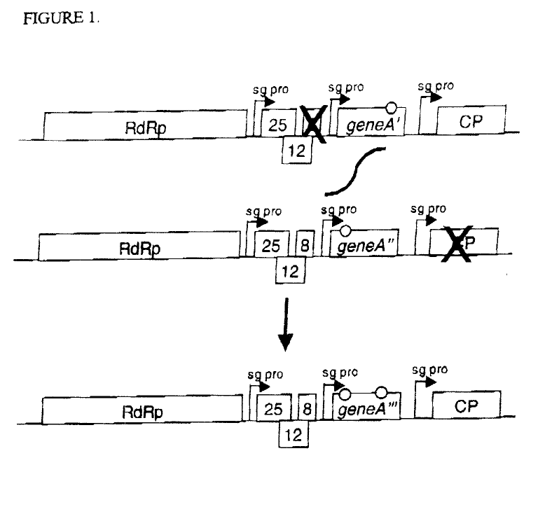

In an embodiment, viral vectors containing complementary mutations in proteins required for systemic spread of the virus are used to introduce variants of the gene of interest. For example, as shown in FIG. 1, a viral vector is constructed including, in the direction of transcription: a RNA-dependent RNA polymerase (RdRp, e.g., from Potato Virus X); essential movement protein encoding sequences under regulatory control of a first subgenomic promoter; a variant of a gene of interest (e.g., an R gene, an Avr gene, etc.) under regulatory control of a second subgenomic promoter; and coat protein under regulatory control of a third subgenomic promoter.

Multiple members of a population of vectors having alternative and complementary mutations in one or the other of a movement protein or a coat protein, each having a variant of the gene of interest, designated “gene A,” are introduced into, e.g., a basal leaf of an intact plant. Only variants that have undergone recombination between the complementary mutations, e.g., in the gene of interest, will be capable of systemic infection and movement throughout the plant. Thus, sampling of distal leaves, e.g., those higher on the plant, provides a simple means of screening and selecting recombined viral vectors. In addition, this technology provides the benefit that recombination and expression are acheived in vivo in a single step.

In one embodiment, RNA recombination via RNA viral vectors is used to create and express combinatorial libraries of shuffled genes. For example, shuffled variants of gene “A” are inserted into vector I, and shuffled variants of gene “B” are inserted into vector II. Vectors I and II have complementary mutations such that only recombinants between the two vectors are capable of movement throughout the plant. A mixed infection of shuffled variants of gene A and gene B is initiated, and recombinant viruses carrying recombined, e.g., shuffled, variants of A and B are recovered from infected, e.g., upper, leaves.

Post-recombination Screening Techniques

Regardless of the diversity generating method or methods employed, identification of novel resistance associated genes and gene products involves one or more screening and/or selection protocol distinguishing nucleic acids encoding products with desired properties. In some instances, the desired property or characteristic relates to the nucleic acid, e.g., hybridization, amplification, or the like. However, in many cases the desired characteristic relates to a functional property conferred by the recombinant nucleic acid, e.g., R gene, Avr gene, pathogenesis related gene, etc., expressed in situ. The following describe exemplary screening modalities that are favorably used in the context of the present invention. In general, the methods permit the identification of productive (i.e., incompatible) interactions between the products of R genes and elicitors. In some cases R genes and genes encoding elicitors, or proteins involved in the synthesis of elicitors, are expressed transiently (or stably) by transfecting cells, most typically plant cells (or tissues or explants, or even whole plants) with recombinant nucleic acids of interest. Alternatively, a plant, or plant explant, or plant cell is exposed to the product of a plant disease response gene and/or an elicitor expressed by, e.g., a plant pathogen. For example, bacterial plant pathogens such as Agrobacterium spp. and Pseudomonas spp. can be favorably employed to introduce recombinant R genes and Avr genes and their products into a host cell or plant of interest for screening purposes. In such cases, the products of the R gene or Avr gene are translocated from the pathogen to the plant cell by the secretory system of the pathogen, e.g., via a Type III secretory system. To facilitate transfer, the recombinant R (or Avr) gene can encode a targeting signal, such as the AvrBs2 or AvrPto target signal sequences. In other alternative methods, a recombinant nucleic acid of interest is expressed in vitro or in vivo, and the product of interest, e.g., an elicitor encoded by, or produced by a biosynthetic enzyme encoded by, a recombinant Avr gene is recovered and then applied to or introduced into a host plant cell for screening.

Resistance to Plant Pathogens

Plant pathogens encompass a broad range of viral, bacterial, fungal, insect and nematode parasites. While the specific resistance exhibited in response to bacterial and fungal pathogens has been the most well characterized to date, the methods of the present invention also provide the means of manipulating the disease response to viral as well as insect and nematode pathogens. Evolved of R genes that, for example, induce reduced SAR, e.g., that lead to decreased phenylpropanoid biosynthesis leading to decreased levels of salicylic acid, can be selected to confer improved resistance to insect pests. It is not necessary, in advance of nucleic acid diversification, e.g., by DNA or RNA shuffling, that either the LRRs responsible for binding to an insect and/or nematode derived elicitor, or the signalling pathway required for activation of an SAR be elucidated. Rather, it is sufficient that an effective means of identifying a productive, i.e., incompatible, interaction be available. One potential indicator of an interaction between a pathogen-derived elicitor and an R gene is resistance to infection by a specified pathogen. Other, biochemical and molecular indicators suitable for screening R genes, including shuffled R genes are described, infra.

Resistance to Infection

Resistance to infection is a multipartite response by the plant, when faced with attempted infection by an avirulent pathogen strain. As described above, adaptive responses include the HR, which isolates the infectious agent, e.g., bacteria or fungus, and SAR, which confers broad resistance through changes in cellular architecture, e.g., lignification, and production of anti-pathogenic and protective substances, e.g., chitinases, β1,3 glucanases, glutathione S-transferase.

At a gross level, two parameters are of significant value in evaluating whether an interaction of a particular Avr/R gene pair results in resistance to infection. Firstly, a decrease in symptoms evaluated at the level of an intact plant, or in isolated tissues or cells provides a desireable means of screening in the context of the present invention. Secondly, a decrease in pathogen growth, e.g., inhibition of cell division, can be profitably used to detect an interaction between incompatible Avr/R genes.

For example, numerous bacterial species infect plants causing various kinds of disease symptoms. Infection by virulent Agrobacterium species results in tumor like growth disturbances such as crown gall, twig gall, cane gall and hairy root. Erwinia sp. result in various blights, wilts and soft rots depending on the species and the host. Pseudomonas sp. result in galls, blights and wilts, as well as leaf spots and canker and bud blast. In addition to leaf spots and certain blights, Xanthomonas sp. result in a variety of rots as well as black venation. As the descriptive titles suggest, the symptoms of the various diseases are readily apparent to and distinguishable by one of skill in the art. A reduction in symptoms can be readily evaluated as a reduction in the number of lesions and/or reduction in the area affected by a described symptom.

Similarly, symptoms resulting from pathogenic viruses and fungi (e.g., of the genus Fusarium, Bremia, etc.) are readily recognizable to those of skill in the art, and can be assessed in the same manner as infections by bacterial pathogens.

Alternatively, a decrease in the growth of a pathogenic organism, or in the number of organisms present as compared to control plants, e.g., lacking an R gene, provides a valuable means of evaluating resistance in a plant host.

Another approach to screening for recombinant nucleic acids encoding proteins with desired properties, e.g., R genes capable of interacting with a specified elicitor, relies on assessment of responses mediated by the protein. For example, interactions between an elicitor and an R protein that lead ultimately to resistance involve, as described above, a hypersensitive response (HR). The programmed cell death that is the hallmark of the HR is readily evaluated either in planta as the localized regions of necrosis surrounded by healthy tissue. Alternatively, cell death corresponding the the HR can be evaluated in cultured plant cells, e.g., isolated plant cells grown in suspension culture by viability staining.

Alternatively, molecular markers of the HR are favorably used to detect interactions between the R proteins and elicitors. As described in more detail above, a variety of changes occur within the plant cell following interaction between cognate resistance gene products and their elicitors. Among the earliest changes are alterations in membrane permeability including electrolyte leakage. Many of these can be measured directly, e.g., electrochemically, osmotically, or indirectly, as changes in turgor associated with electrolyte loss. For example, measurement of calcium flux can be performed on isolated cells or tissues using fluorescent analogs of common calcium chelators such as EGTA, e.g., fura-2, indo-1, and other commercially available probes: Molecular Probes, Eugene, Oreg. Fluorescent labels for other biologically relevant ions, e.g., Na⁺, K⁺, Cl⁻, etc., are also commercially available from this and other sources, for use in the context of the current invention.

As a further consequence of R protein activation, downstream signalling pathways are activated, including among others, both lipases, e.g., EDS1 and kinases, e.g., Pto, Pti1 of tomato, MAPK homologs of tobacco, and the like. Activation of serine/threonine and/or tyrosine kinases, e.g., by phosporylation resulting in increased kinase activity towards a designated substrate, is another useful screening tool in the context of the present invention. For example, using the purified substrate myelin basic protein (MBP), activity of certain serine/threonine kinases including numerous mitogen activated protein kinases (MAPK) can be determined.

Pathogenesis Related Gene Induction

Following interaction between an elicitor and the LRR domain of an R protein, activation of second messenger signalling cascades leads to the induction of a multitude of pathogenesis related (PR) gene products. Many such genes, e.g., PR genes of many species including tobacco and tomato, myb1, Rnase NE, hsr203J, CHS, CHI and IFR genes of alfalfa, and the like, have been cloned. Induction of expression at a transcriptional level consistent with induction of the HR can be measured according to numerous methods available in the art, including northern analysis and dot blotting of RNA and RT-PCR.

Similarly, accumulation of products associated with resistance, such as various pathogenesis related proteins described, supra, as well as other proteins associated with the HR and SAR, can be measured to determine whether an incompatible interaction or other specified activity dependent on properties exhibited, e.g., by the products of shuffled R genes or Avr genes has taken place. In some cases, it will be desirable to measure accumulation of proteins, e.g., phenylalanine amonia lyase (PAL) by such molecular techniques as western analysis. In other cases, it will be preferred to assess accumulation of proteins indirectly by the products of their activity, e.g., callose production by β1,3 glucanases.

Another means for identifying interactions between R/AVR products involves the use of reporter gene fusions operably linked to promoters such as PR-1, hsr203J, known to be induced in response to R gene activation. In general, it is preferable to use readily visualized reporters such as β-glucuronidase (GUS), green fluorescent protein (GFP, including mutant GFPs), and luciferase. For example, PR-1-GUS, and hsr203J-GUS promoter fusions have been utilized to provide a readily visible means to elucidate agents and timing of gene induction related to resistanse responses (Beffa et al. (1995) EMBO J 14:5753; Pontier et al. (1994) Plant J 5:507).

Bio-detectors

The R genes with novel specificities and improved signalling characteristics of the invention provide the basis for biological detectors of plant pathogens and other environmental stressors. An important feature of the LRR domains of R genes is their structural plasticity that enables them to interact with ligands possessing various structural and functional characteristics. In the present invention, nucleic acid diversification, e.g., shuffling and selection methods are utilized to evolve R genes that act as sensors to detect a wide variety of environmentally relevant ligands. One class of ligands are components of crop pathogens of interest. Such components include known elicitor and elicitor related molecules, as well as pathogen derived products that have not been ascribed elicitor function. Other classes of ligands include molecules, whether protein or peptide gene products, or small molecules produced by a plant in response to environmental stressors such as heat, drought, uv irradiation, and wounding. Other ligands include but are not limited to human and animal pathogens as well as other chemical ligands.

A reporter placed under the regulatory control of a gene triggered in the SAR or HR pathway, e.g., a PR promoter facilitates detection of the interaction between the R protein and the ligand associated with the pathogen or environmental state of interest. Structural genes encoding visible reporters such as GFP or luciferase are examples of reporters favorably used in the context of the present invention, as are proteins with enzymatic activites that convert a chromogenic substrate to a readily visualizable products (e.g., GUS, β-GAL, or an enzyme involved in carotenoid biosynthesis, e.g., phytoene synthase). Additionally, regulatory genes such as the C and R (anthocyanin regulatory) loci of maize that are involved in the induction of anthocyanin production in plants can also be used as markers.

While the recognition aspect of the recognition-to-activation pathways induced by R genes involves the LRR domain, by evolving the kinase domains, it is possible to alter or modulate the signalling pathway, and hence, the repertoire of genes induced in response to ligand/R protein interaction.

The evolved R genes with novel recognition properties can be stably intgrated into plant genomes by any method known in the art, e.g., microinjection, electroporation, agrobacterium mediated transformation, biolistics. Alternatively, such R genes can be delivered by viral vectors as described herein, and in PCT/US00/32298 “SHUFFLING OF AGROBACTERIUM AND VIRAL GENES, PLASMIDS AND GENOMES FOR IMPROVED PLANT TRANSFORMATION” by Castle and Lassner, filed Nov. 22, 2000, which is incorporated by reference herein in its entirety.

Conferring Resistance to Plant Pathogens Using R/Avr Genes

The utilization of viral vectors to introduce R genes with specified characteristics related to the detection of ligands, or to the activation of response pathways provides a means to confer resistance to pathogens and other stresses upon plants growing in the field. This is particularly beneficial, when the pathogen (or pathogens) are discovered after planting, and for which the plants do not have endogenous disease recognition abilities. For example, an R gene with the ability to interact with a specified elicitor or pathogen can be cloned into a plant viral vector selected for its infectivity in the plant species of interest. Numerous resources for determining plant susceptibility are available and known to those of skill in the art, see, e.g., Brunt et al. (1996) Viruses of Plants: Descriptions and Lists from the VIDE Database C.A.B. International, U. K. Once a suitable virus has been identified and a non-pathogenic vector produced, e.g., by deletion of coat protein or movement protein encoding sequences, the vector is utilized to introduce R genes with desired characteristics (e.g., ability to activate response pathways upon exposure to a specified elicitor) into target plants in situ. The vector can be introduced, e.g., by mechanical inoculation, micro-injection into the plant's vasculature, etc., into the target plants. Upon infection, viral subgenomic promoters activate expression of the R gene. In the presence of the pathogen or other elicitor, the introduced R gene activates the disease resistance response pathways thus conferring resistance to the pathogen. As previously described, in some circumstances, it is desirable to employ R genes with altered signalling capabilities, e.g., with altered kinase domains, that preferentially modulate alternative response programs.

In addition to transfection of annual crop plants, such methods provide novel means of protecting perrennial and woody species, including important agricultural (e.g., grapevine, fruit trees, etc.), horticultural (e.g., rose, rhododendron, azealea, etc.) and ornamental and commercial tree species (e.g., oak, maple, chestnut, elm, pine, cedar, etc.) from emerging and existing pathogens. For example, the methods of the present invention can be used to produce novel recombinant R genes which interact productively with elicitors produced, e.g., by bacterial pathogens such as Xylella fastidiosa and Xanthomonas capestris, the causative agents of Pierce's disease and Oak shoot blight, respectively, and by fungal pathogens such as the causative agents of Oak wilt “anthracnose,” Dutch elm disease, and Chestnut Blight (i.e., Cryptocline cinerscens, Ophiostoma ulmi, and Cryphonectria parasitica. For example, according to the methods described herein, recombinant R genes produced by any of the recombination or mutagenesis procedures discussed above (or any combination thereof) using known or newly isolated parental sequences, can be screened for their ability to interact with isolated or expressed elicitors derived from, or cultures of, X. fastidiosa bacteria. Libraries of recombinant R genes can be introduced into host plant explants, e.g., grapevine leaf disc explants, or into test species such as Arabidopsis, using a viral vector as described herein (e.g., via mechanical inoculation using a vector such as Arabis mosaic noepovirus that is capable of infecting both species). The transiently transfected plants or plant explants (or plant cells) are then exposed to an endogenous or exogenous source of the relevant elicitor, e.g., by cotransfecting with an Avr gene, in the case of a known and isolated Avr gene, or by exposing or infecting the test plant to the pathogen, or, e.g., extracts produced therefrom. Following screening, for example by a reporter mediated assay, such as the ability of the recombinant R gene product to induce expression of a visible reporter (such as GFP), virus incorporating the identified R genes is recovered. These viral vectors, or an alternative viral vector, depending on test and host species compatibility, can then be used to introduce the recombinant R gene with novel desired binding properties into the target host species, e.g., into grapevine root stock by micro-injection into the vasculature of the growing plant.

Vectors for Expressing R Genes in Plants

Virus-based expression vectors are powerful tools to produce high level expression of foreign genes inplant cells or plant tissues. They are also amenable to high throughput screening methods, expecially when a visible phentoype (such as hypersensitive cell1 death) is available. Examples of such vectors include those modified from RNA viruses such as TMV, PVX and TRV and those from DNA viruses such as geminiviruses. The specific genes to be tested can be inserted into the viral genome and expressed from a viral subgenomic promoter. Infectious DNA or RNA transcripts of the virus containing the test genes are made in vitro and used to inoculate plant cells. The inoculated cells can be either cultured cells (suspension culture or protoplasts) or intact tissue (detached leaves or whole plants). Specific interaction between R genes and elicitors, e.g., Avr gene products, triggers the HR reaction resulting in cell death and other defense responses. Cell death can be assayed by viability staining in cell culture, or in the case of intact tissue, it can be visualized by local lesions on the leaves. In many cases it is desirable to assay for defense responses that occur prior to cell death, such as activation of defense related genes, calcium flux or electroyte leakage. The virus from individual lesions can be rescued and evaluated further. Alternatively, viral RNA can be extracted and characterised allowing identification of genes of interest. Using this method, one can test the function of R genes and their variants, or Avr genes and their variants or both. For example, either one or both of the R genes and the Avr genes (and respective variants) can be introduced into a plant or plant cell (or tissue or explant) where the respective gene products are expressed cytoplasmically. A plant defense response is then detected by any of the methods described above, permitting identification of functional interactions between variant R and Avr genes. In some cases, Avr genes are integrated into the genome of the host plant which is then inoculated with a viral vector carrying R genes to be evaluated. Conversely, R genes can be inserted into plant genome and the viral vector used to deliver and express Avr genes. Alternatively, the R gene or Avr gene can be introduced into the plant or plant cell by infecting the plant with a plant pathogen such as a bacterial pathogen, e.g., Agrobacterium spp., Pseudomonas spp. In some cases, it is preferable to employ a non-infectious microorganism for this purpose.

Plant Viruses

Expression of foreign genes in plants has been demonstrated with a number of viral vector systems. Viruses are typically useful as vectors for expressing exogenous DNA sequences in a transient manner in plant hosts. In contrast to agrobacterium mediated transformation which results in the stable integration of DNA sequences in the plant genome, non-integrating viral vectors are generally replicated and expressed in the cytoplasm of the plant cell without the need for chromosomal integration. Plant virus vectors offer a number of advantages, specifically: DNA copies of viral genomes can be readily manipulated in E. coli, and transcribed in vitro, where necessary, to produce infectious RNA copies; naked DNA, RNA, or virus particles can be easily introduced into mechanically wounded leaves of intact plants; high copy numbers of viral genomes per cell results in high expression levels of introduced genes; common laboratory plant species as well as monocot and dicot crop species are readily infected by various virus strains; infection of whole plants permits repeated tissue sampling of single library clones; recovery and purification of recombinant virus particles is simple and rapid; and because replication occurs without chromosomal insertion, expression is not subject to position effects. These many advantages are exploited by the present invention, firstly: for the introduction and expression of R genes and Avr genes, including recombinant, e.g., shuffled, R/Avr genes, and secondly: as a tool for RNA based recombination.

For example, Hammond-Kosack et al. (MPMI (1995) 8:181) reported that expression of the Clodosporium fulvum avr9 gene from a potato virus X vector leads to hypersensitive cell death mediated by the tomato Cf9 gene. Rommens et al. (Plant Cell (1995) 7:249) used the PVX vector system to to characterize a tomato PTO resisistance gene homolog which confers sensitivity to the herbicide fenthion. Tobias et al (Plant J (1999) 17:41) also employed a PVX vector containing the avirulence gene AvrPto to elicit a resistance response in tomato and N. benthamiana plants harboring the Pto gene.

Over six-hundred-fifty plant viruses have been identified, many of which are suitable as vectors in the present invention, depending on the host plant species of interest. Plant viruses are known which infect every major food-crop, as well as most species of horticultural interest. The host range varies between viruses, with some viruses infecting a broad host range (e.g., alfalfa mosaic virus infects more than 400 species in 50 plant families) while others have a narrow host range, sometimes limited to a single species (e.g. barley yellow mosaic virus).

Approximately 75% of the known plant viruses have genomes which are single-stranded (ss) messenger sense (+) RNA polynucleotides. Major taxonomic classifications of ss-RNA(+) plant viruses include the bromovirus, capillovirus, carlavirus, carmovirus, closterovirus, comovirus, cucumovirus, fabavirus, furovirus, hordeivirus, ilarvirus, luteovirus, potexvirus, potyvirus, tobamovirus, tobravirus, tombusvirus, trichovirus, and many others. Other plant viruses exist which have single-stranded antisense (−) RNA (e.g., rhabdoviridae), double-stranded (ds) RNA (e.g., cryptovirus, reoviridae), or ss or ds DNA genomes (e.g., geminivirus and caulimovirus, respectively).

Plant viruses can be engineered as vectors to accomplish a variety of functions including introducing R genes and/or Avr genes or other pathogenesis related genes into plants. Examples of both DNA and RNA viruses have been used as vectors for gene replacement, gene insertion, epitope presentation and complementation, (see, e.g., Scholthof, Scholthof and Jackson, (1996) “Plant virus gene vectors for transient expression of foreign proteins in plants,” Annu.Rev.of Phytopathol. 34:299-323). including viruses selected from among: an alfamovirus, a bromovirus, a capillovirus, a carlavirus, a carmovirus, a caulimovirus, a closterovirus, a comovirus, a cryptovirus, a cucumovirus, a dianthovirus, a fabavirus, a fijivirus, a furovirus, a geminivirus, a hordeivirus, a ilarvirus, a luteovirus, a machlovirus, a maize chlorotic dwarf virus, a marafivirus, a necrovirus, a nepovirus, a parsnip yellow fleck virus, a pea enation mosaic virus, a potexvirus, a potyvirus, a reovirus, a rhabdovirus, a sobemovirus, a tenuivirus, a tobamovirus, a tobravirus, a tomato spotted wilt virus, a tombusvirus, and a tymovirus.

Methods for the transformation of plants and plant cells using sequences derived from plant viruses include the direct transformation techniques described herein relating to DNA molecules, see e.g., Jones, ed. (1995) Plant Gene Transfer and Expression Protocols, Humana Press, Totowa, N.J., for a recent compilation. In addition viral sequences can be cloned adjacent T-DNA border sequences and introduced via Agrobacterium mediated transformation, or “Agroinfection.”

Viral particles comprising the plant virus vectors of the invention can also be introduced by mechanical inoculation using techniques well known in the art, (see e.g., Cunningham and Porter, eds. (1997) Methods in Biotechnology, Vol.3. Recombinant Proteins from Plants: Production and Isolation of Clinically Useful Compounds, for detailed protocols). Briefly, for experimental purposes, young plant leaves are dusted with silicon carbide (carborundum), then inoculated with a solution of viral transcript, or encapsidated virus and gently rubbed. Large scale adaptations for infecting crop plants are also well known in the art, and typically involve mechanical maceration of leaves using a mower or other mechanical implement, followed by localized spraying of viral suspensions, or spraying leaves with a buffered virus/carborundum suspension at high pressure. Alternatively, viruses can be introduced into woody species (e.g., trees, grapevine, etc.) by “micro-injection” into the vasculature (or cambium) of the plant. Any of these techniques can be adapted to the present invention, and are useful for alternative applications depending on the choice of plant virus, and host species, as well as the scale of the specific transformation application.

Molecular Biology

General texts which describe molecular biological techniques useful herein, including the use of vectors, promoters and many other relevant topics related to, e.g., the cloning and expression of R genes, Avr genes, pathogenesis related genes and viral sequences, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)). Similarly, examples of techniques sufficient to direct persons of skill through in vitro amplification methods, including the polymerase chain reaction (PCR) the ligase chain reaction (LCR), Qβ-replicase amplification and other RNA polymerase mediated techniques (e.g., NASBA), e.g., for the production of the homologous nucleic acids of the invention are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40kb are generated. One of skill will appreciate that essentially any RNA can be converted into a double stranded DNA suitable for restriction digestion, PCR expansion and sequencing using reverse transcriptase and a polymerase. See, Ausubel, Sambrook and Berger, all supra.

The present invention also relates to host cells and organisms which are transformed with vectors of the invention, and the production of polypeptides of the invention, e.g., R proteins, eliciting, and other proteins and polypeptides encoded by exogenous DNAs, by recombinant techniques. Host cells are genetically engineered (i.e., transformed, transduced or transfected) with the vectors of this invention, which may be, for example, a cloning vector or an expression vector. The vector may be, for example, in the form of a plasmid, an agrobacterium, a virus, a naked polynucleotide, or a conjugated polynucleotide. The vectors are introduced into plant tissues, cultured plant cells or plant protoplasts by standard methods including electroporation (From et al., Proc. Natl. Acad. Sci. USA 82, 5824 (1985), infection by viral vectors such as cauliflower mosaic virus (CaMV) (Hohn et al., Molecular Biology of Plant Tumors, (Academic Press, New York, 1982) pp. 549-560; Howell, US 4,407,956), high velocity ballistic penetration by small particles with the nucleic acid either within the matrix of small beads or particles, or on the surface (Klein et al., Nature 327, 70-73 (1987)), use of pollen as vector (WO 85/01856), or use of Agrobacterium tumefaciens or A. rhizogenes carrying a T-DNA plasmid in which DNA fragments are cloned. The T-DNA plasmid is transmitted to plant cells upon infection by Agrobacterium tumefaciens, and a portion is stably integrated into the plant genome (Horsch et al., Science 233, 496-498 (1984); Fraley et al., Proc. Natl. Acad. Sci. USA 80, 4803 (1983)).

The engineered host cells can be cultured in conventional nutrient media modified as appropriate for such activities as, for example, activating promoters or selecting transformants. These cells can optionally be cultured into transgenic plants. Plant regeneration from cultured protoplasts is described in Evans et al., “Protoplast Isolation and Culture,” Handbook of Plant Cell Cultures 1, 124-176 (MacMillan Publishing Co., New York, 1983); Davey, “Recent Developments in the Culture and Regeneration of Plant Protoplasts,” Protoplasts, (1983) pp. 12-29, (Birkhauser, Basal 1983); Dale, “Protoplast Culture and Plant Regeneration of Cereals and Other Recalcitrant Crops,” Protoplasts (1983) pp. 31-41, (Birkhauser, Basel 1983); Binding, “Regeneration of Plants,” Plant Protoplasts, pp. 21-73, (CRC Press, Boca Raton, 1985).

The present invention also relates to the production of transgenic organisms, which may be bacteria, yeast, fungi, or plants. A thorough discussion of techniques relevant to bacteria, unicellular eukaryotes and cell culture may be found in references enumerated above and are briefly outlined as follows. Several well-known methods of introducing target nucleic acids into bacterial cells are available, any of which may be used in the present invention. These include: fusion of the recipient cells with bacterial protoplasts containing the DNA, electroporation, projectile bombardment, and infection with viral vectors (discussed further, below), etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, a plethora of kits are commercially available for the purification of plasmids from bacteria. For their proper use, follow the manufacturer's instructions (see, for example, EasyPrep™, FlexiPrep™, both from Pharmacia Biotech; StrataClean™, from Stratagene; and, QIAprep™ from Qiagen). The isolated and purified plasmids are then further manipulated to produce other plasmids, used to transfect plant cells or incorporated into Agrobacterium tumefaciens related vectors to infect plants. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (all supra). A catalogue of Bacteria and Bacteriophages useful for cloning is provided, e.g., by the ATCC, e.g., The ATCC Catalogue of Bacteria and Bacteriophage (1992) Gherna et al. (eds) published by the ATCC. Additional basic procedures for sequencing, cloning and other aspects of molecular biology and underlying theoretical considerations are also found in Watson et al. (1992) Recombinant DNA Second Edition Scientific American Books, NY.

Transforming Nucleic Acids into Plants.

An aspect of the invention pertain to the production of transgenic plants comprising R and Avr genes of the invention. Techniques for transforming plant cells with nucleic acids are generally available and can be adapted to the invention by the use of plasmids, viruses, and components thereof, and by the use of agrobacterium strains comprising R genes, Avr genes, PR genes and the like. In addition to Berger, Ausubel and Sambrook, useful general references for plant cell cloning, culture and regeneration include Jones (ed) (1995) Plant Gene Transfer and Expression Protocols—Methods in Molecular Biology, Volume 49 Humana Press Towata N.J.; Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods Springer Lab Manual, Springer-Verlag (Berlin Heidelberg New York) (Gamborg). A variety of cell culture media are described in Atlas and Parks (eds) The Handbook of Microbiological Media (1993) CRC Press, Boca Raton, Fla. (Atlas). Additional information for plant cell culture is found in available commercial literature such as the Life Science Research Cell Culture Catalogue (1998) from Sigma-Aldrich, Inc (St Louis, Mo.) (Sigma-LSRCCC) and, e.g., the Plant Culture Catalogue and supplement (1997) also from Sigma-Aldrich, Inc (St Louis, Mo.) (Sigma-PCCS). Additional details regarding plant cell culture are found in Croy, (ed.) (1993) Plant Molecular Biology Bios Scientific Publishers, Oxford, U.K.

The nucleic acid constructs of the invention, e.g., plasmids, viruses, DNA and RNA polynucleotides, are introduced into plant cells, either in culture or in the organs of a plant by a variety of conventional techniques. To use artificially evolved, e.g., shuffled, sequences, recombinant DNA or RNA vectors suitable for transformation of plant cells are isolated and/or prepared. To introduce an exogenous DNA, which can be an artificially evolved DNA, the exogenous DNA sequence can be incorporated into an appropriate vector and transformed into the plant as indicated above. Where the sequence is expressed, the sequence is optionally combined with transcriptional and translational initiation regulatory sequences which direct the transcription or translation of the sequence from the exogenous DNA in the intended tissues of the transformed plant.

Where DNA vectors are selected, the DNA constructs of the invention, for example plasmids, or naked or variously conjugated-DNA polynucleotides, (e.g., polylysine-conjugated DNA, peptide-conjugated DNA, liposome-conjugated DNA, etc.) can be introduced directly into the genomic DNA of the plant cell using techniques such as electroporation and microinjection of plant cell protoplasts, or the DNA constructs can be introduced directly to plant cells using ballistic methods, such as DNA particle bombardment.

Microinjection techniques for injecting e.g., cells, embryos, and protoplasts, are known in the art and well described in the scientific and patent literature. For example, a number of methods are described in Jones (ed) (1995) Plant Gene Transfer and Expression Protocols—Methods in Molecular Biology, Volume 49 Humana Press Towata N.J., as well as in the other references noted herein and available in the literature.

For example, the introduction of DNA constructs using polyethylene glycol precipitation is described in Paszkowski, et al., EMBO J. 3:2717 (1984). Electroporation techniques are described in Fromm, et al., Proc. Nat'l. Acad. Sci. USA 82:5824 (1985). Ballistic transformation techniques are described in Klein, et al., Nature 327:70-73 (1987). Additional details are found in Jones (1995) supra.

Regeneration of Transgenic Plants

Transformed plant cells which are derived by any of the above transformation techniques can be cultured to regenerate a whole plant which possesses the transformed genotype and thus the desired phenotype, e.g., resistance to a designated pathogen, or interaction with a specified elicitor. Such regeneration techniques rely on manipulation of certain phytohormones in a tissue culture growth medium, optionally relying on a biocide and/or herbicide marker which has been introduced together with the desired nucleotide sequences. Plant regeneration from cultured protoplasts is described in Evans, et al., Protoplasts Isolation and Culture, Handbook of Plant Cell Culture, pp. 124-176, Macmillian Publishing Company, New York, (1983); and Binding, Regeneration of Plants, Plant Protoplasts, pp. 21-73, CRC Press, Boca Raton, (1985). Regeneration can also be obtained from plant callus, explants, somatic embryos (Dandekar, et al., J. Tissue Cult. Meth. 12:145 (1989); McGranahan, et al., Plant Cell Rep. 8:512 (1990)), organs, or parts thereof. Such regeneration techniques are described generally in Klee, et al., Ann. Rev. of Plant Phys. 38:467-486 (1987). Additional details are found in Payne (1992) and Jones (1995), both supra. These methods are adapted to the invention to produce transgenic plants using evolved vectors, including agrobacteria and viruses containing the R and/or Avr genes of the invention.

Preferred plants for the transformation and expression of the novel R and/or Avr genes, as well as other constructs of this invention include agronomically and horticulturally important species. Such species include, but are not restricted to members of the families: Graminae (including corn, rye, triticale, barley, millet, rice, wheat, oats, etc.); Leguminosae (including pea, beans, lentil, peanut, yam bean, cowpeas, velvet beans, soybean, clover, alfalfa, lupine, vetch, lotus, sweet clover, wisteria, and sweetpea); Compositae (the largest family of vascular plants, including at least 1,000 genera, including important commercial crops such as sunflower); Vitaceae (e.g., grapevine) and Rosaciae (including raspberry, apricot, almond, peach, rose, etc.), as well as nut plants (including, walnut, pecan, hazelnut, etc.), and ornamental and forest trees (including Cornus, Ulmus, Pinus, Quercus, Pseutotsuga, Sequoia, Populus,etc.)

Additionally, preferred targets for transformation by the nucleic acids of the invention include, as well as those specified above, plants from the genera: Agrostis, Allium, Antirrhinum, Apium, Arachis, Asparagus, Atropa, Avena (e.g., oats), Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuca, Fragaria, Geranium, Glycine, Helianthus, Heterocallis, Hevea, Hordeum (e.g., barley), Hyoscyamus, Ipomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza (e.g., rice), Panicum, Pelargonium, Pennisetum (e.g., millet), Petunia, Pisum, Phaseolus, Phleum, Poa, Prunus, Ranunculus, Raphanus, Ribes, Ricinus, Rubus, Saccharum, Salpiglossis, Secale (e.g., rye), Senecio, Setaria, Sinapis, Solanum, Sorghum, Stenotaphrum, Theobroma, Trifolium, Trigonella, Triticum (e.g., wheat), Vicia, Vigna, Vitis, Zea (e.g., corn), and the Olyreae, the Pharoideae and many others. As noted, plants in the family Graminae are a particularly preferred target plants for the methods of the invention.

Common crop plants which are targets of the present invention include corn, rice, triticale, rye, cotton, soybean, sorghum, wheat, oats, barley, millet, sunflower, canola, peas, beans, lentils, peanuts, yam beans, cowpeas, velvet beans, clover, alfalfa, lupine, vetch, lotus, sweet clover, wisteria, sweetpea and nut plants (e.g., walnut, pecan, etc).

In construction of recombinant expression cassettes of the invention, which include, for example, helper plasmids comprising virulence functions, and plasmids or viruses comprising exogenous DNA sequences such as structural genes, a plant promoter fragment is optionally employed which directs expression of a nucleic acid in any or all tissues of a regenerated plant. Examples of constitutive promoters include the cauliflower mosaic virus (CaMV) 35S transcription initiation region, the 1′- or 2′-promoter derived from T-DNA of Agrobacterium tumefaciens, and other transcription initiation regions from various plant genes known to those of skill. Alternatively, the plant promoter may direct expression of the polynucleotide of the invention in a specific tissue (tissue-specific promoters) or may be otherwise under more precise environmental control (inducible promoters). Examples of tissue-specific promoters under developmental control include promoters that initiate transcription only in certain tissues, such as fruit, seeds, or flowers.

Any of a number of promoters which direct transcription in plant cells can be suitable. The promoter can be either constitutive or inducible. In addition to the promoters noted above, promoters of bacterial origin which operate in plants include the octopine synthase promoter, the nopaline synthase promoter and other promoters derived from native Ti plasmids. See, Herrara-Estrella et al. (1983), Nature, 303:209-213. Viral promoters include the 35S and 19S RNA promoters of cauliflower mosaic virus. See, Odell et al. (1985) Nature, 313:810-812. Other plant promoters include the ribulose-1,3-bisphosphate carboxylase small subunit promoter and the phaseolin promoter. The promoter sequence from the E8 gene and other genes may also be used. The isolation and sequence of the E8 promoter is described in detail in Deikman and Fischer, (1988) EMBO J. 7:3315-3327. Many other promoters are in current use and can be coupled to an exogenous DNA sequence to direct expression of the nucleic acid.

If expression of a polypeptide, including pathogen- and plant-derived gene products, such as R proteins, eliciting, biosynthetic enzymes, and reporters of the present invention, is desired, a polyadenylation region at the 3′-end of the coding region is typically included. The polyadenylation region can be derived from the natural gene, from a variety of other plant genes, or from, e.g., T-DNA.

In some cases, the vector comprising the sequences (e.g., promoters or coding regions) from genes encoding expression products and transgenes of the invention will optionally include a nucleic acid subsequence, a marker gene which confers a selectable, or alternatively, a screenable, phenotype on plant cells. For example, the marker may encode biocide tolerance, particularly antibiotic tolerance, such as tolerance to kanamycin, G418, bleomycin, hygromycin, or herbicide tolerance, such as tolerance to chlorosluforon, or phosphinothricin (the active ingredient in the herbicides bialaphos or Basta). See, e.g., Padgette et al. (1996) “New weed control opportunities: Development of soybeans with a Round UP Ready™ gene” In: Herbicide-Resistant Crops (Duke, ed.), pp 53-84, CRC Lewis Publishers, Boca Raton (“Padgette, 1996”). For example, crop selectivity to specific herbicides can be conferred by engineering genes into crops which encode appropriate herbicide metabolizing enzymes from other organisms, such as microbes. See, Vasil (1996) “Phosphinothricin-resistant crops” In: Herbicide-Resistant Crops (Duke, ed.), pp 85-91, CRC Lewis Publishers, Boca Raton) (“Vasil”, 1996).

In some applications stable and vertical transmission of an R gene of other nucleic acid of the invention is desired. One of skill will recognize that after the exogenous DNA sequence is stably incorporated in transgenic plants and confirmed to be operable, it can be introduced into other plants by sexual crossing. Any of a number of standard breeding techniques can be used, depending upon the species to be crossed.

While the foregoing invention has been described in some detail for purposes of clarity and understanding, it will be clear to one skilled in the art from a reading of this disclosure that various changes in form and detail can be made without departing from the true scope of the invention. For example, all the techniques, methods, compositions, apparatus and systems described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method for identifying a plant disease resistance gene with a specified characteristic, the method comprising:
(a) providing a plurality of disease resistance (R) gene segments;
(b) recombining the plurality of R gene segments, thereby producing a library of recombinant R gene segments;
(c) optionally repeating the recombination of steps (a) and (b) one or more times;
(d) expressing at least one recombinant R gene segment in at least one plant cell, and exposing the at least one plant cell to an elicitor of a plant defense response; and
(e) detecting at least one plant defense response, thereby identifying a plant disease resistance (R) gene with a specified characteristic.

2. The method of claim 1, further comprising repeating the recombination and screening process of steps (a) through (e) at least one additional time.

3. The method of claim 1, the R gene segments comprising at least one nucleic acid sequence selected from among a disease resistance gene of tomato, rice, Arabidopsis, barley, corn, soybean, flax, sugar beet and wheat.

4. The method of claim 1, the R gene segments comprising at least one nucleic acid sequence selected from among homologs of Bs2, Cf2, Cf4, Cf5, Cf9, Dm3, Fen, Hcr2, Hcr9, Hs1^{pro-1}, I2, L6, LRK10, M, Mlo, Mi, N, Pib, PRF, Pti1, Pto, Rp1-D, RPM1, RPP, RPS2, RPS4, Rx, Xa1 and Xa21.

5. The method of claim 1, further comprising mutating one or more of the segments provided in (a).

6. The method of claim 1, comprising recombining the population of R gene segments in vivo, in vitro or in silico.

7. The method of claim 6, comprising recombining RNA viruses comprising R gene segments in vivo.

8. The method of claim 7, comprising recombining RNA viruses comprising R gene segments in plant cells.

9. The method of claim 1, wherein expressing the at least one recombinant R gene segment comprises stably integrating the at least one recombinant R gene operably linked to a promoter functional in a plant cell into the genome of the at least one plant cell.

10. The method of claim 1, wherein expressing the at least one recombinant R gene segment comprises inoculating the at least one plant cell with a non-integrating viral vector comprising the at least one recombinant R gene.

11. The method of claim 10, wherein the non-integrating viral vectors comprise (+) strand RNA viruses, (−) strand RNA viruses, ambisense viruses, single stranded DNA viruses or double stranded DNA viruses.

12. The method of claim 11, wherein the non-integrating viral vector is selected from a tobamovirus, a potexvirus, a potyvirus, a tobravirus or a geminivirus.

13. The method of claim 11, wherein expression of the R genes is regulated by at least one viral or non-viral promoter active in the plant cell.

14. The method of claim 13, wherein the promoter is a viral subgenomic promoter.

15. The method of claim 1, wherein expressing the at least one recombinant R gene segment comprises infecting the at least one plant cell with a plant pathogen comprising the at least one recombinant R gene.

16. The method of claim 15, wherein the plant pathogen is a bacterial plant pathogen.

17. The method of claim 16, wherein the bacterial plant pathogen is a species of Pseudomonas.

18. The method of claim 15, wherein the R gene segment further comprises a targeting signal.

19. The method of claim 17, wherein the target signal comprises an AvrBs2 or an AvrPto target signal.

20. The method of claim 1, comprising exposing the at least one plant cell to an elicitor of a plant defense response comprising a product of an Avr gene or Avr gene homolog.

21. The method of claim 20, comprising exposing the at least one plant cell to an Avr gene product produced by a plant pathogen.

22. The method of claim 21, wherein the Avr gene product produced by the plant pathogen is a heterologous Avr gene product.

23. The method of claim 20, comprising exposing the at least one plant cell to an Avr gene product produced by a non-pathogenic microorganism or virus.

24. The method of claim 23, wherein the virus is a non-integrating viral vector.

25. The method of claim 20, comprising exposing the at least one plant cell to an Avr gene product produced by the plant cell.

26. The method of claim 25, wherein the plant cell is a transgenic plant cell expressing an Avr gene.

27. The method of claim 1, comprising detecting at least one plant defense response comprising a hypersensitive (HR) response, a systemic aquired resistance (SAR) response, an induction of genes associated with a HR or a SAR, an accumulation of gene products or compounds associated with a HR or a SAR or a resistance to an infection by a plant pathogen.

28. The method of claim 28, comprising detecting resistance to an infection by a plant pathogen comprises detecting a decrease in symptoms or a decrease in pathogen growth.

29. The method of claim 27, wherein the plant pathogen is a bacterial, fungal, insect or nematode pathogen.

30. The method of claim 27, comprising detecting a plant defense response by one or more of viability staining, visualization of local lesions, measuring calcium flux or monitoring electrolyte leakage.

31. The method of claim 1, wherein the specified characteristic is selected from among ligand binding, downstream signalling and kinase activation.

32. The method of claim 1, further comprising recovering at least one R gene with a specified characteristic.

33. The method of claim 32, comprising recovering the at least one R gene by at least one of PCR, LCR, Qβ amplification, cloning, isolation of an RNA transcript and reverse transcription.

34. The method of claim 33, wherein the RNA transcript is a viral RNA transcript.

35. The method of claim 32, further comprising integrating the at least one R gene with a specified characteristic operably linked to a promoter functional in a plant cell into the genome of a plant cell.

36. The method of claim 35, further comprising regenerating the plant cell, thereby producing a transgenic plant that expresses a product of the R gene with a specified characteristic.

37. The method of claim 36, further comprising exposing the transgenic plant to at least one elicitor.

38. The method of claim 37, wherein the elicitor is the product of a recursively recombined Avr gene or Avr gene homolog, or a recursively recombined gene encoding an enzyme catalyzing production of an elicitor.

39. The method of claim 37, detecting at least one plant defense response, thereby identifying an elicitor with a desired property.

40. The method of claim 39, wherein the desired property is interacting with the product of the R gene with a specified characteristic.

41. A transgenic plant produced by the method of claim 36.

42. A method of conferring resistance to at least one plant pathogen by introducing the R gene with a specified characteristic of claim 32 into a plant or plant cell.

43. The method of claim 42, comprising introducing the R gene by inoculating the plant or plant cell with a non-integrating viral vector comprising the R gene with a specified characteristic.

44. The method of claim 42, comprising stably integrating the R gene with a specified characteristic operably linked to a promoter functional in a plant into a plant cell, and regenerating the plant cell comprising the R gene with a specified characteristic into a transgenic plant.

45. A method for identifying an elicitor of a plant defense response with a desired property, the method comprising:
(a) providing a plurality of nucleic acid segments comprising at least one elicitor or enzyme catalyzing production of an elicitor of a plant disease response;
(b) recombining the plurality of nucleic acid segments, thereby producing a library of recombinant nucleic acids encoding elicitors or enzymes catalyzing production of elicitors;
(c) optionally repeating the recombination of steps (a) and (b) one or more times;
(d) exposing at least one plant cell to at least one elicitor encoded by or produced by an enzyme encoded by a member of the library of recombinant nucleic acids of step (b); and
(e) detecting at least one plant defense response, thereby identifying at least one elicitor with a desired property.

46. The method of claim 45, further comprising repeating the recombination and screening process of steps (a) through (e) at least one additional time.

47. The method of claim 45, the plurality of nucleic acid segments of step (a) comprising at least one nucleic acid sequence comprising a viral nucleic acid sequence, a bacterial nucleic acid sequence, a fungal nucleic acid sequence, an insect nucleic acid or a nematode nucleic acid.

48. The method of claim 45, the plurality of nucleic acid segments of step (a) comprising at least one nucleic acid sequence selected from an Avr gene or Avr gene homolog.

49. The method of claim 45, comprising recombining the plurality of nucleic acids in vivo, in vitro or in silico.

50. The method of claim 49, comprising recombining RNA viruses comprising at least one elicitor or enzyme catalyzing production of an elicitor of a plant disease response in vivo.

51. The method of claim 50, comprising recombining RNA viruses comprising at least one elicitor or enzyme catalyzing production of an elicitor of a plant disease response in plant cells.

52. The method of claim 45, comprising exposing the at least one plant cell to at least one elicitor by externally applying the at least one elicitor to the at least one plant cell.

53. The method of claim 45, comprising exposing the at least one plant cell to at least one elicitor by inoculating the at least one plant cell with a non-integrating viral vector comprising a member of the library of recombinant nucleic acids encoding elicitors or enzymes catalyzing production of elicitors.

54. The method of claim 53, wherein the non-integrating viral vector comprises (+) strand RNA viruses, (−) strand RNA viruses, ambisense RNA viruses, single stranded DNA viruses or double stranded DNA viruses.

55. The method of claim 54, wherein the non-integrating viral vector is selected from a tobamovirus, a potexvirus, a potyvirus, a tobravirus or a geminivirus.

56. The method of claim 54, wherein expression of the elicitor or enzyme catalyzing an elicitor is regulated by at least one viral or non-viral promoter active in the plant cell.

57. The method of claim 56, wherein the promoter is a viral subgenomic promoter.

58. The method of claim 45, comprising exposing the at least one plant cell to at least one elicitor by infecting the at least one plant cell with a plant pathogen comprising a member of the library of recombinant nucleic acids encoding elicitors or enzymes catalyzing production of elicitors.

59. The method of claim 58, wherein the plant pathogen is a bacterial plant pathogen.

60. The method of claim 59, wherein the bacterial plant pathogen is a species of Pseudomonas.

61. The method of claim 45, wherein the at least one plant cell comprises a cultured plant cell, a plant protoplasts, a plant tissue, an isolated plant organ, an intact plant organ or a whole plant.

62. The method of claim 61, wherein the at least one plant cell expresses an R gene with a specified characteristic.

63. The method of claim 62, wherein the at least one plant cell comprises a transgenic plant cell.

64. The method of claim 63, wherein the R gene with a specified characteristic is a recursively recombined R gene.

65. The method of claim 45, comprising detecting a plant defense response selected from among a plant disease response, a hypersensitive (HR) response, and a systemic aquired resistance (SAR) response, induction of a gene associated with a HR or SAR, an accumulation of gene products or compounds associated with a HR or SAR, or a resistance to an infection.

66. The method of claim 45, comprising detecting a plant defense response by one or more of viability staining, visualization of local lesions, measuring calcium flux or monitoring electrolyte leakage.

67. The method of claim 45, wherein the desired property is selected from among binding properties, response elicitation.

68. The method of claim 45, further comprising recovering at least one nucleic acid encoding an elicitor with a desired property or an enzyme catalyzing production of an elicitor with a desired property.

69. The method of claim 68, comprising recovering the at least one nucleic acid by at least one of PCR, LCR, Qβ amplification, cloning, isolation of an RNA transcript and reverse transcription.

70. The method of claim 69, wherein the RNA transcript is a viral RNA transcript.

71. A method of inducing a plant defense response by exposing at least one plant cell to the elicitor with a desired property of claim 45.

72. The method of claim 71, comprising inoculating the at least one plant cell with a non-integrating viral vector comprising a nucleic acid encoding the elicitor with a desired property.

73. A method for identifying a functional interaction between a plant disease resistance gene and an elicitor, the method comprising:
(i) introducing a first viral vector comprising a plant disease resistance (R) gene, and a second viral vector comprising a gene encoding an elicitor or enzyme catalyzing production of an elicitor into at least one plant cell, such that the R gene and the elicitor are cytoplasmically expressed in the at least one plant cell; and
(ii) detecting at least one plant defense response, thereby identifying a functional interaction between the R gene and the elicitor.

74. The method of claim 1, wherein the viral vectors comprise non-integrating viral vectors selected from among (+) strand RNA viruses, (−) strand RNA viruses, ambisense RNA viruses, single stranded DNA viruses and double stranded DNA viruses.

75. The method of claim 74, wherein the non-integrating viral vector is selected from a tobamovirus, a potexvirus, a potyvirus, a tobravirus or a geminivirus.

76. The method of claim 74, wherein expression of the R genes is regulated by at least one viral or non-viral promoter active in the plant cell.

77. The method of claim 76, wherein the promoter is a viral subgenomic promoter.

78. The method of claim 73, wherein at least one of the R gene or the gene encoding an elicitor or enzyme catalyzing production of an elicitor is a member of a library of genes or gene segments, which library comprises one or more of a genomic library, an expression library, a transcript library, a DNA library, an RNA library, a PCR amplicon library, an EST library, a mutant library and a recursively recombined library.

79. The method of claim 73, wherein at least one of the R gene or the gene encoding an elicitor or enzyme catalyzing production of an elicitor comprise recursively recombined genes.

80. The method of claim 73, wherein the population of plant cells comprises cultured plant cells, plant protoplasts, plant tissues, isolated plant organs, intact plant organs or whole plants.

81. The method of claim 73, comprising detecting a plant defense response selected from among a plant disease response, a hypersensitive (HR) response, a systemic aquired resistance (SAR) response, an induction of genes associated with a HR or SAR response, an accumulation of gene products or compounds associated with a HR or SAR response, a resistance to infection by a plant pathogen, a decrease in symptoms of an infection, and a reduction in pathogen growth.

82. The method of claim 73, comprising detecting a plant defense response by one or more of viability staining, visualization of local lesions, measuring calcium flux or monitoring electrolyte leakage.

83. A method for identifying a functional interaction between a plant disease resistance gene and an elicitor, the method comprising:
(i) exposing at least one plant cell to a plant pathogen comprising an elicitor of a plant defense response and a plant disease resistance (R) gene; and
(ii) detecting at least one plant defense response, thereby identifying a functional interaction between the R gene and the elicitor.

84. The method of claim 83, wherein the plant pathogen comprises a bacterial plant pathogen.

85. The method of claim 84, wherein the bacterial plant pathogen is a species of Pseudomonas.

86. The method of claim 83, wherein the plant disease resistance (R) gene is a member of a library of genes or gene segments, which library comprises one or more of a genomic library, an expression library, a DNA library, A PCR amplicon library, an EST library, a mutant library and a recursively recombined library.

87. The method of claim 83, wherein a product of the R gene is translocated from the pathogen to the plant cell by a secretory system of the pathogen.

88. The method of claim 87, wherein the secretory system of the pathogen comprises a Type III secretory system.

89. The method of claim 83, wherein the R gene segment further comprises a targeting signal.

90. The method of claim 87, wherein the target signal comprises an AvrBs2 or an AvrPto target signal.

91. The method of claim 83, wherein the population of plant cells comprises cultured plant cells, plant protoplasts, plant tissues, isolated plant organs, intact plant organs or whole plants.

92. The method of claim 83, comprising detecting a plant defense response selected from among a plant disease response, a hypersensitive (HR) response, a systemic aquired resistance (SAR) response, an induction of genes associated with a HR or SAR response, an accumulation of gene products or compounds associated with a HR or SAR response, a resistance to infection by a plant pathogen, a decrease in symptoms of an infection, and a reduction in pathogen growth.

93. The method of claim 83, comprising detecting a plant defense response by one or more of viability staining, visualization of local lesions, measuring calcium flux or monitoring electrolyte leakage.

94. A bio-detector comprising:
(i) an R gene encoding a product capable of activation by at least one elicitor; and
(ii) a reporter operably linked to a promoter responsive to the activated product of the R gene.

95. The bio-detector of claim 83, wherein the R gene comprises a recursively recombined R gene with a specified characteristic.

96. The bio-detector of claim 95, wherein the R gene encodes a product capable of activation by a designated elicitor.

97. The bio-detector of claim 96, wherein the designated elicitor is an Avr gene product.

98. The bio-detector of claim 83, wherein the reporter comprises a green fluorescent protein (GFP), a carotenoid biosynthetic enzyme, an anthocyanin regulatory gene or a luciferase.

99. The bio-detector of claim 83, wherein the promoter comprises a promoter derived from a gene in a systemic aquired resistance (SAR) pathway.

100. The bio-detector of claim 83, wherein the promoter comprises a PR promoter.

101. A plant or plant cell comprising the bio-detector of claim 83.

102. The plant or plant cell of claim 101, wherein one or more component of the bio-detector is stably integrated into a chromosome.

103. The plant or plant cell of claim 101, wherein one or more component of the bio-detector is extrachromosomally replicated.

104. The plant or plant cell of claim 103, wherein the one or more extrachromosomally replicated component of the bio-detector comprises a non-integrating viral vector.

105. A method for producing a gene with a desired property, the method comprising:
(a) introducing a plurality of RNA viral vectors comprising one or more gene of interest into at least one cell;
(b) growing the cell under conditions permitting cytoplasmic recombination between the plurality of RNA viral vectors, thereby producing a library of recombinant RNA viral vectors;
(c) optionally recovering at least one recombinant viral vector and repeating steps (a) and (b);
(d) identifying at least one RNA viral vector comprising a gene with a desired property.

106. The method of claim 105, comprising introducing the plurality of RNA viral vectors by inoculating at least one cell with infectious viral transcripts.

107. The method of claim 105, comprising introducing the plurality of RNA viral vectors by introducing a plurality of cDNA molecules corresponding to viral transcripts.

108. The method of claim 107, wherein viral transcripts comprising the plurality of cDNA molecules are produced in the cytoplasm of the at least one cell.

109. The method of claim 107, wherein the plurality of cDNA molecules are introduced by electroporation, microinjection, biolistics, agrobacterium mediated transformation or agroinfection.

110. The method of claim 105, wherein the RNA viral vector comprises a plant viral vector.

111. The method of claim 110, wherein the RNA viral vector is selected from among a tobamovirus, a potyvirus, a tobravirus and a potexvirus.

112. The method of claim 110, wherein the RNA viral vector comprises a Tobacco Mosaic Virus (TMV), a TMV homolog or an engineered viral vector derived from a TMV or TMV homolog.

113. The method of claim 105, wherein the gene of interest comprises a protein coding sequence.

114. The method of claim 105, wherein the at least one cell comprises a plant cell.

115. The method of claim 114, wherein the plant cell comprises an isolated plant cell, a protoplast, a plant explant, a plant tissue or an intact plant.

116. The method of claim 114, comprising growing the plant cell in suspension culture.

117. The method of claim 114, comprising growing at least one intact plant comprising the plant cell.

118. The method of claim 105, wherein the cytoplasmic recombination is mediated by template switching of an RNA polymerase expressed by the at least one cell.

119. The method of claim 118, wherein the RNA polymerase is a plant viral RNA polymerase.

120. The method of claim 118, wherein the RNA polymerase is a mutant or engineered viral RNA polymerase that enhances the frequency of homologous or non-homologous RNA recombination relative to a wild-type plant viral RNA polymerase.

121. The method of claim 120, wherein the mutant or engineered viral RNA polymerase is produced by a directed evolution process.

122. The method of claim 121, wherein the directed evolution process comprises a DNA or RNA recombination procedure.

123. The method of claim 105, comprising recovering at least one recombinant viral vector by isolating RNA from the at least one cell.

124. The method of claim 105, comprising identifying the at least one RNA viral vector comprising a gene with a desired property by selection or screening.

125. The method of claim 105, comprising introducing at least a first RNA viral vector incapable of systemic infection in a plant and a second RNA viral vector incapable of systemic infection in a plant, which first and second viral vectors have complementary mutations in genes essential for systemic infection, and identifying at least one recombinant RNA viral vector by selecting or screening for RNA viral vectors capable of systemic infection.

126. The method of claim 125, wherein the genes having complementary mutations comprise one or more of a gene encoding a viral movement protein or a gene encoding a viral coat protein.

127. The method of claim 125, wherein selecting or screening is performed by sampling a plant cell or tissue remote from the site of introduction.
